(19) United States
(12) Patent Application Publication
(10) Pub. No.: US 2015/0017066 A1
(43) Pub. Date: Jan. 15, 2015

(54) LINE IMMUNOASSAY TESTING DEVICE

(71) Applicant: Immco Diagnostics, Inc., Buffalo, NY (US)

(72) Inventors: Kishore MALYAVANTHAM, Williamsville, NY (US); Lakshmanan SURESH, Williamsville, NY (US); William MAGGIO, Buffalo, NY (US)

(21) Appl. No.: 14/326,718

(22) Filed: Jul. 9, 2014

Related U.S. Application Data
(60) Provisional application No. 61/844,110, filed on Jul. 9, 2013.

(51) Int. Cl. G01N 33/53; B23K 26/38

(52) U.S. Cl. CPC G01N 33/5302; B23K 26/38
USPC 422/69; 219/121.72

(57) ABSTRACT

A testing device has a base and multiple strips connected to the base. Multiple antigens are placed on each strip. The testing device or strips may be affixed to a sheet under a shield. Fluid can be applied to the testing device or the strips. The testing device can be used for biochemical testing, such as line immunoassay (LIA) testing.

### CROSS-REFERENCE TO RELATED APPLICATIONS

This application claims priority to the provisional patent application entitled “Line Immunoassay Testing Device and Method of Use,” filed Jul. 9, 2013 and assigned U.S. App. No. 61/844,110, the disclosure of which is hereby incorporated by reference.

### FIELD OF THE INVENTION

This invention relates to line immunoassay (LIA) testing and, more particularly, to an LIA testing device and method of manufacture.

### BACKGROUND OF THE INVENTION

As many as 23.5 million Americans may suffer from an autoimmune disease and such diseases are rising in prevalence. Autoimmune diseases are chronic and can be life-threatening. Approximately 80-100 different autoimmune diseases have been identified and at least 40 additional diseases are suspected to have an autoimmune basis. Symptoms can cross many specialties and can affect any organs of the body. These autoimmune diseases are difficult to detect and initial symptoms are often intermittent or non-specific until the autoimmune disease is in an acute stage. Consequently, doctors and specialists may be unaware of the interrelationships among various autoimmune diseases.

LIA is an alternative to Western Blots and Dot Blots. LIA, which is also known as or uses a similar methodology as line blots, dot blots, or line assays, is a biochemical test that measures the presence, absence, or amount of a macromolecule in a solution through the use of an antibody or immunoglobulin. The macromolecule detected by the immunoassay is often referred to as an “analyte.” In many instances, this macromolecule is a specific protein or a nucleic acid. Analytes in biological liquids such as serum, blood, saliva, urine, or other bodily fluids of a human or animal are frequently measured using immunoassays for medical and research purposes. LIA may be used for autoimmune, infectious, or metabolic disease diagnosis or for other testing, research, or diagnosis.

There is a need to customize testing devices for LIA. Custom dimensions and customized numbers of antigens may be needed to meet specific requirements. Manufacturing custom testing devices can be cumbersome using traditional die cutting based manufacturing methods. The design of the various testing devices can be expensive and the testing devices can have high failure rates due to, for example, dull blades or worn dies.

Furthermore, many LIA testing devices lack traceability. Narrow dimensions of the various testing device components can hinder registration or application of traceability information.

What is needed is an improvement to LIA testing and, more particularly, an improved LIA testing device and method of use.

### BRIEF SUMMARY OF THE INVENTION

In a first embodiment, a testing device is provided. The testing device may be configured for LIA testing. The testing device has a base and multiple strips connected to the base. There may be perforations defined between the base and the strips proximate an end of each strip closest to the base.

Each of the strips has a long dimension and a short dimension defining a surface.

Multiple distinct antigens are included on the surface of each strip. Each antigen is located on each strip at the same position along the long dimension. Each strip may contain at least three different controls. In an example, the strip has four different controls.

Opposite of the base, each strip also may have an identification feature on the surface. This identification feature may be a code unique to the strip that the identification feature is disposed on.

Each strip also may have multiple testing areas. Each testing area contains at least one antigen or at least two antigens.

In a second embodiment, a testing system is provided. The testing system includes a testing device and a sheet. The testing device may be configured for LIA testing. The testing device has a base and multiple strips connected to the base. Each of the strips has a long dimension and a short dimension defining a surface. Multiple distinct antigens are included on the surface of each strip. Each antigen is located on each strip at the same position along the long dimension.

The sheet has a shield. At least the strips are configured to be affixed to the sheet under the shield. The sheet may have an adhesive strip that affixes the strips to the sheet. The shield may be permanently affixed to the sheet along at least one edge of the shield. The sheet also may include a table on a surface of the sheet with the shield.

In a third embodiment, a method is provided. The method includes receiving a sheet of inert material that has a plurality of antigen locations and control locations and receiving a number of strips to be formed in the sheet and dimensions for the strips. The inert material may be plastic. Gaps are cut in the sheet with a laser to form the number of strips with the dimensions. The strips are connected to a base. Each strip has a long dimension and a short dimension that define a surface. The antigen locations and control locations are disposed on the surface of each strip. A positional order of the antigen locations and control locations is identical along the long dimension of each strip. An identification feature is formed on the surface of each of the strips using the laser. The identification feature may be a code that is unique to the strip. Perforations may be formed in the sheet between the strips and the base using the laser. The testing device that is formed using this method may be configured for LIA.

### DESCRIPTION OF THE DRAWINGS

For a fuller understanding of the nature and objects of the invention, reference should be made to the following detailed description taken in conjunction with the accompanying drawings, in which:

FIG. 1 is a top view of a testing device;

FIG. 2 is a top view of a sheet configured for use in conjunction with the testing device of FIG. 1;

FIG. 3 is a top view of an embodiment of the sheet illustrated in FIG. 2 with parts of the testing device from FIG. 1;

FIG. 4 is a flowchart of an embodiment of a method of using a testing device;

FIG. 5 is a flowchart of another embodiment of a method of using a testing device; and

FIG. 6 is a flowchart of an embodiment of a method of manufacturing a testing device.

### DETAILED DESCRIPTION OF THE INVENTION

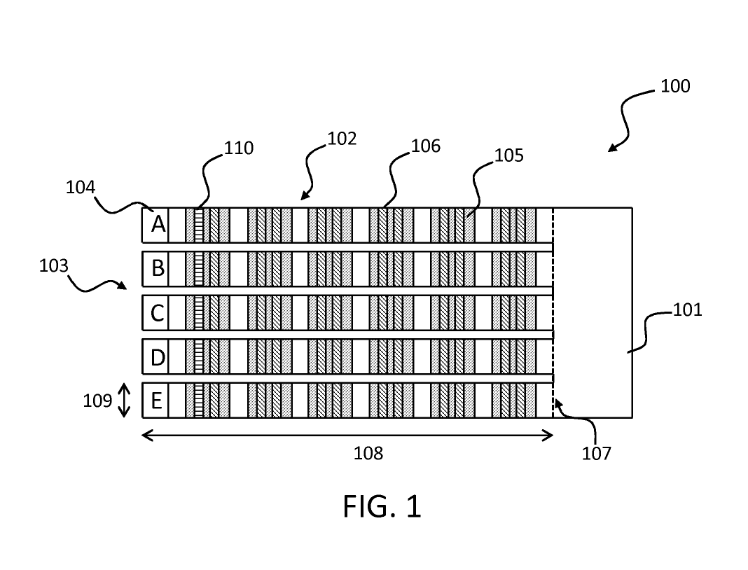

FIG. 1 is a top view of a testing device 100 according to an embodiment of the present invention. In FIG. 1, the testing device 100 has a base 101 and multiple strips 102 connected to or extending from the base 101. Parts of the strips 102 are separated by a gap 103. The strips 102 and base 101 may be fabricated of plastic or some other inert material. Examples of plastics that can be used for the strips 102 and base 101 include natural or synthetic polymers such as polyester, polystyrene, polyethylene terephthalate (PET), or other plastics. A laser, such as a CO₂ laser, may be used to form the gaps 103. Other techniques for forming the gaps 103 will be apparent in light of the present disclosure. Thus, in one instance, the testing device 100 may be manufactured from a single, rectangular piece of plastic. The plastic or other inert material may be flexible. At least some portions of the testing device 100 can be transparent, semitransparent, or colored.

In an embodiment, the strips 102 have a long dimension 108 of approximately 11.5 cm and a short dimension 109 of approximately 2.5 mm.

An identification feature 104 is located on each strip 102 opposite of the base 101. FIG. 1 uses letters A-E, but the identification feature 104 may be or include other information. The identification feature 104 can include one or more letters or numbers or can contain a 2D or 3D barcode. For example, the identification feature 104 can be a code that is unique to the corresponding strip 102. The identification feature 104 can be formed using the same laser that formed the gaps 103. In one instance, the laser that formed the gaps 103 can etch or fabricate the identification feature 104 onto or into the testing device 100. Other techniques for forming the identification feature 104 will be apparent in light of the present disclosure.

In an embodiment, the identification feature 104 can include a panel abbreviation, a lot number, and a serial number. The panel abbreviation may be a three letter abbreviation. The lot number may be a seven digit number. The serial number may be a four digit serial number. Of course, other forms of the identification feature 104 are possible and these are listed merely as examples. The identification feature 104 can be modified to suit a particular application or dimensions of the strip 102.

The identification feature 104 can be used to link a strip 102 to, for example, a particular patient, sample, fluid source, technician, or laboratory. Such a link may be performed either electronically or manually. With respect to the identification feature 104, the code on the strip 102 can be entered into, for example, a Laboratory Information Management System (LIMS) or a barcode can be scanned into a LIMS. A camera or scanner also can retrieve the identification feature 104 during processing of the strips 102 and this retrieved identification feature 104 can be linked to a LIMS. Once linked, the identification feature 104 or test results associated with an identification feature 104 can be used by various software packages or during results analysis.

Each strip 102 includes multiple testing areas 105 (illustrated by shading in FIG. 1). Six testing areas 105 are illustrated on each strip 102, but this is merely for ease of illustration. Other numbers of testing areas 105 are possible. Each testing area 105 may contain at least one antigen location 106, which may be immobilized on the testing area 105. While two antigen locations 106 are illustrated on each testing area 105 in FIG. 1, more or fewer antigen locations 106 may be on each testing area 105. In one embodiment, some testing areas 105 have one antigen location 106 and other testing areas 105 have two or more antigen locations 106. For example, three antigen locations 106 can be placed on one testing area 105. Capture reagents may be used instead of antigens in the antigen locations 106 in some instances.

The testing areas 105 can be selected and fabricated from a variety of materials such as different derivatives of nitrocellulose (including nitrocellulose material with various surfactants and various porosities imparting different properties), polyvinylidene fluoride (PVDF) membranes of different porosities and hydrophobic or hydrophilic and binding characteristics toward the selected antigens, or derivatives of other polymers. The thickness of the testing area 105 relative to the thickness of the strip 102 can vary depending on the type of testing area 105 used and how the testing area 105 is fabricated. For example, the testing area 105 may be laminated with adhesives on one side so that the testing area 105 can bind to polystyrene backing. The thickness of the testing areas 105 may not affect test procedures if the volume of the test sample is sufficient to submerge the strip 102.

As seen in FIG. 1, each of the strips 102 has a long dimension 108 and a short dimension 109 defining a surface of the testing device 100 on which the testing areas 105 are located. The antigen locations 106 are approximately parallel to each other along the short dimension of each strip 102. In embodiments, multiple antigen locations 106 are included on each strip 102, and the same antigens are located at the same positions along the long dimension 108 of each of the strips 102. Therefore, the antigen attached to the antigen location 106 nearest the base 101 will be the same antigen on each strip 102. Other antigens will typically be positioned at the same point along the long dimension 108 of each strip 102 at the antigen locations 106. The distinct antigens attached to the antigen locations 106 form a panel of markers to which, for example, antibodies that are specific for the antigens, if present in a patient sample that is applied to the strips 102, will bind to the antigens thereby immobilizing the antibodies and facilitating their detection using suitable reagents and device(s) as further described hereon.

Each strip 102 may have multiple antigen locations 106 (illustrated with hatch marks in FIG. 1). Each strip 102 also may have at least one control location 110 which may substitute for or be used in addition to the antigen locations 106. The positions of the antigen locations 106 relative to the control location 110 can vary depending on the application. For example, a strip 102 may have between 4 and 24 antigen locations 106, some of which may be substituted with control locations 110. The control aids in interpreting results. For example, the testing device 100 may comprise a control used as a reference against which other indicators may be compared. In another example, the testing device 100 may comprise a control that is a procedural control, for confirmation that a process step was performed (e.g., indicating the presence of serum). Other types of controls, and combinations of controls, can be used. In one particular embodiment, each strip 102 contains at least one, two, three, or four different controls.

Biological molecules used as antigens or controls in the context of the test strips 102 are prepared in buffers at a desired concentration and applied to an immunoreactive surface that is capable of permanently binding these molecules. This immunoreactive surface may be part of the antigen locations 106 and can be a derivative of nitrocellulose or other polymers such as, for example, PVDF. The immunoreactive surface can be fabricated as cards suitable for a batch process, as a continuous roll suitable for in-line processing, or other methods. Cards and rolls of the immunoreactive surface can be manufactured by custom slitting and laminating. The antigen or control molecule solutions are dispensed on to the immunoreactive surface using equipment such as, for example, Bio Dot RR120 or other models designed for batch or in-line use.

In one embodiment, a first control location comprises a colored line. In an embodiment, the colored line is used as a so-called blocking control in that the colored line dissolves into solution so that it is no longer visible, thus providing visual evidence that, for example, the strip 102 was properly submerged in a blocking solution. In embodiments, the colored line comprises a dye, examples of which include but are not limited to FD&C Blue No. 1, also known as Brilliant Blue FCF. Other dyes can be used to form other colored lines and can be used either alone or in combinations. Such dyes include but are not limited to FD&C Red 40, FD&C Red 3, FD&C Yellow 5, and in addition to the aforementioned blue colored line, can provide red, green, orange or other shades suitable for use in the control line of the first control location.

In embodiments, a second control location is provided and is considered to be a conjugate control location. The conjugate control comprises a concentration of immunoglobulin, such as IgG, and is used to confirm that the detection aspect of the immunodiagnostic test is functioning. For example, in one embodiment, a detection antibody which is conjugated to, for example, an enzyme that catalyzes a reaction which produces a detectable signal, is contacted with the IgG in the conjugate control location. In an embodiment, a detection antibody is conjugated with a horseradish peroxidase enzyme. If the assay is functioning properly, the horseradish peroxidase-conjugated detection antibody will bind to the IgG in the conjugate control location, and upon exposure to a suitable chromogenic or chemiluminescent substrate, many of which are known in the art, will produce a detectable signal, thus confirming that hybridization between the detection antibody and pre-loaded Ig occurred, and that the enzymatic detection process functioned. Those skilled in the art will recognize that the same approach to determining the presence or absence of autoantibodies that are bound to the antigen in the antigen locations 106 can be used in a similar manner, i.e., by using a detection antibody to bind to immobilized autoantibodies at the antigen location 106 and to produce a detectable signal.

In embodiments, a third control location is provided and is considered to be a sample or serum control. This control comprises anti-human Ig and thus serves to provide an indication as to whether or not a patient sample was contacted with the strip 102. In particular, it is expected under ordinary circumstances that serum tested using the strip 102 will comprise Ig that are not autoantibodies. Such Ig will bind to the anti-human Ig in the serum control and are detected using any suitable technique, such as the same or similar detection approach as described for the conjugate control location, i.e., by using a detection antibody which produces a detectable signal.

In embodiments, a fourth control location is provided and is considered to be a “cut-off” control. The cut-off control is a control location that comprises a lesser concentration of human Ig relative to the amount of Ig in the conjugate control location, and is used in the same way as the conjugate control, meaning it serves as a substrate for detection antibodies. The cut-off control provides a background amount of Ig and thus establishes a threshold amount of signal below which a detectable signal is considered to be a consequence of background signal. Thus, the detectable signal from the cut-off control location can be used to compare to and/or normalize signal from antigen locations 106, thereby decreasing recordation of false-positive results, or for deeming the test result to be indeterminate. In embodiments, the amount of Ig in the conjugate control location is at least one, or at least two orders of magnitude greater than the amount of Ig in the cut-off control location. In embodiments, the conjugate control location comprises human IgG at 25-50 nanograms/microliter, the cut-off control location comprises human IgG at 0.25-2.0 nanograms/microliter, and the serum control location comprises anti-human IgG at 25-50 nanograms/microliter. Depending on the product and kit components, such as enzyme conjugate and substrate used, the control lines dispense at a rate of between approximately 0.5 to 2 ul per cm of immunoreactive surface, which results in lines with expected reactivity. If an enzyme conjugate solution strength is increased or decreased, the concentration of control line biomolecules is appropriately decreased or increased, respectively, to produce the expected immunoreactivity.

The testing device 100 may include perforations 107 at the end of or proximate the end of each strip 102 closest to the base 101. Thus, the strips 102 can be manually separable from the base 101 at the perforations. In embodiments where a laser formed the gaps 103, the same laser also can be used to form the perforations 107.

The number of testing areas 105, antigen locations 106, and control locations 110 can vary. This is illustrated in Table 1.

TABLE 1

| | Number of | Number of | Number of |
|---|---|---|---|
| | testing | antigen | control |
| Product or Panel | areas 105 | locations 106 | locations 110 |
| ANA (Anti-nuclear | 11 | 17 | 3 |
| antibody) panel | | | |
| ANA Advanced panel | 13 | 21 | 3 |
| Myositis panel | 9 | 14 | 3 |
| Liver panel | 7 | 9 | 3 |
| Hearing Loss Panel | 3 | 1 | 3 |
| ANA (Anti-nuclear | 11 | 17 | 3 |
| antibody) panel - | | | |
| evaluation kit | | | |
| ANA Advanced panel - | 13 | 21 | 3 |
| evaluation kit | | | |
| Myositis panel - | 9 | 14 | 3 |
| evaluation kit | | | |
| Liver panel - | 7 | 9 | 3 |
| evaluation kit | | | |

The number of strips 102 on a testing device 100 can vary between various panels by use of the laser that forms the gaps 103. The long dimension 108 and short dimension 109 can remain constant even though the number of strips 102 can vary. Alternatively, the long dimension 108 or short dimension 109 can vary between different panels. In one specific example, the long dimension 108 is shortened and the number of antigen locations 106 or control locations 110 is lowered to decrease costs.

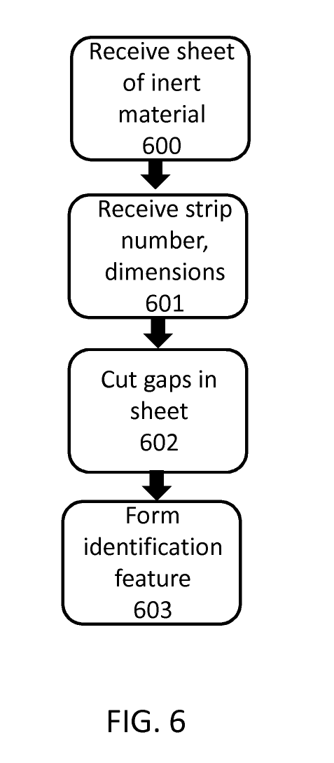

FIG. 6 is a flowchart of an embodiment of a method of manufacturing a testing device. In the embodiment of FIG. 6, the testing device 100 is formed from a sheet of inert material with antigen locations 106 and control locations 110 already formed thereon. The testing areas 105 also may already be formed on the sheet. The inert material can be, for example, plastic. At 600, a system that includes a laser receives a sheet. At 601, the system that includes a laser receives a number of and dimensions of the strips 102 to be formed in the sheet. At 602, the laser cuts gaps 103 in the sheet to form the strips 102. The strips 102 may be part of a testing device, such as the testing device 100 illustrated in FIG. 1. At 603, the laser forms an identification feature 104 on each of the strips 102. The dimensions and number of strips 102 can vary, which enables flexibility during manufacturing to accommodate multiple testing device 100 designs or configurations. In an example, between one and five polystyrene or polymer sheets with assembled immunoreactive antigen coated areas are placed on the laser bed for processing. These sheets will result in 100 to 500 strips 102 in sets of twenty, which can be packaged into kits. The CO₂ laser engravers used to form the strips 102 may be manufactured by, for example, Tortech, Epilog, York Laser, JQ Laser, Vision, or Laser Systems. The laser engraver can have laser bed dimensions to accommodate the sheets, X, Y, and Z axis motors for the laser head, sufficient wattage for the CO₂ laser to perform the cutting operation, or other features such as vision-based inspection systems.

The total number of antigens and controls can be varied based on various testing requirements. Different panels or products have variable number of antigen or test lines. In the examples described in Table 1, the number of control lines is kept constant. However, the number of control lines can change based on testing requirements. Each testing area 105 can have one or two antigen locations 106 or control locations 110, but additional lines can be incorporated. Using the design described here, 14 testing areas 105 can accommodate a total of 28 antigen locations 106 or control locations 110. This can further by increased if both sides of the strip 102 are used for reactions. For example, 56 antigen locations 106 or control locations 110 can be incorporated in a strip with an 11.5 cm long dimension 108 if both sides of the strip 102 are used. If additional lines are incorporated into each testing area 105, the total number of antigen locations 106 or control locations 110 that can be incorporated on a strip 102 can double to 104. Thus, the design allows an additional number of antigen locations 106 or control locations 110 in a strip 102.

If both sides of the strip 102 are used for reactions, the identification feature 104 may only be present on one surface of the strip 102.

The testing device 100 processing involves a biological fluid specimen, such as blood, urine, saliva, serum, plasma, cerebrospinal fluid, tissue extracts, or other biological fluids, being applied to the testing device 100. These other biological fluids may come from a human or non-human animal, but also may be some other fluids known to those skilled in the art. The fluid specimen is tested to determine if it comprises a macromolecule that specifically binds antigen(s) in the antigen locations 106. In general, the macromolecule that binds to the antigen will be an auto-antibody, which is subsequently detected using a detection antibody according to techniques known in the art and as described above for the control locations 110. The testing device 100 may be incubated with a sample or fluid specimen followed by several steps of washing and sequential incubations with assay reagents during testing. The detectable reaction between the autoantibodies in the fluid, the antigens 106 and detection antibodies can be either qualitative or quantitative. Reactions are read visually and can be reported as positive, negative, or indeterminate. Indeterminate may mean that the results had a comparable intensity to the testing area (such as antigen location 106 or control location 110), strip 102, or cut-off line.

In an example, between 10 and 20 strips 120 are packaged in a kit for use in LIA testing. Other numbers of strips 102 can also be provided in a kit.

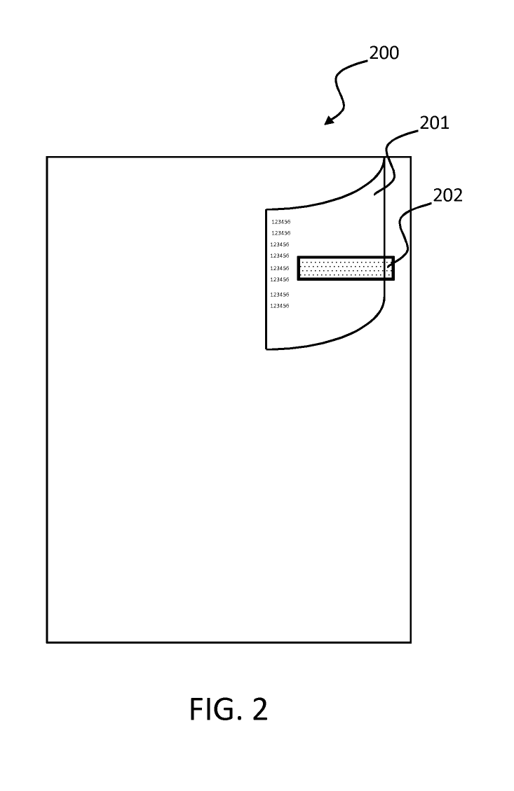

FIG. 2 is a top view of a sheet 200 configured for use in conjunction with the testing device 100 of FIG. 1. The sheet 200 has a shield 201 and an adhesive strip 202. This adhesive strip 202 enables the testing device 100 or strips 102 to be affixed to the sheet 200 under the shield 201. The shield 201 protects the testing device 100 or strips 102 during handling or processing. The shield 201 is clear, colored, or tinted plastic in one instance and may be attached or affixed to the sheet 200 along at least one edge of the shield 201. This shield 201 can be permanently attached or affixed to the sheet 200 along this edge. The shield 201 can be repeatedly lifted from the sheet 200 except at the edge that is attached or affixed to the sheet 200. The adhesive strip 202 may have a permanent adhesive between the adhesive strip 202 and the sheet 200 and a reusable adhesive between the adhesive strip 202 and the testing device 100, strips 102, or shield 201. The permanent adhesive in one embodiment can cause damage to the sheet 200 if removed. The reusable adhesive may have a lower adhesive strength than the permanent adhesive. Thus, the testing device 100, strips 102, or shield 201 can be repeatedly lifted from the adhesive strip 202 without becoming permanently attached and without causing significant damage to the testing device 100, strips 102, or shield 201. The reusable adhesive may be configured such that peeling or lifting the shield 201 does not result in visible residue sticking to the shield 201. Other adhesive strengths or adhesive designs are possible in keeping with the current disclosure. In another embodiment, the adhesive strip 202 has two different adhesive strengths on the two opposite sides with the weaker adhesive strength facing the shield 201.

The strips 102 or testing device 100 can be aligned under the shield 201. For example, the strips 102 or testing device 100 may be aligned using markers, lines, or other visual cues on the sheet 200.

This sheet 200 can be used for multiple sessions. Different testing devices 100 or strips 102 can be replaced under the shield 200 during various sessions to interpret or examine the results. This enables the sheet 200 to be used for multiple archiving, scanning, or analysis sessions.

The sheet 200 may further include slots or grids to place or hold the individual strips 102 of the testing device 100. The sheet 200 may further include reference information or a key on the sheet 200 under the shield 201 or proximate the shield 201, such as next to the location of the testing device 100. Other tables or reference information also may be included on the sheet 200. The strips 102 of the testing device 100 may be separated at the perforations 107 and placed on the sheet 200 in one embodiment. In an alternate embodiment, the entire testing device 100 is placed on the sheet 200.

The sheet 200 may be used to preserve or archive data. A camera or scanner may be used to read the sheet 200 or the strips 102 of the testing device 100 on the sheet 200. The sheet 200 is configured to be compatible for scanning and analysis by a device such as, for example, a camera, tray scanner, or flatbed scanner. A user can interpret the sheet 200 or testing device 100 and, for example, mark boxes or portions of tables on the sheet 200. During or after imaging or scanning, this information may be recorded or automatically digitized by an optical character recognition (OCR) software engine. Results of the scanning may communicated to a processor. Reporting software may be used to interpret, store, or present the results. The sheet 200 may be destroyed in some instances once the information contained on it is analyzed or stored. This may eliminate the need for storing biohazardous materials.

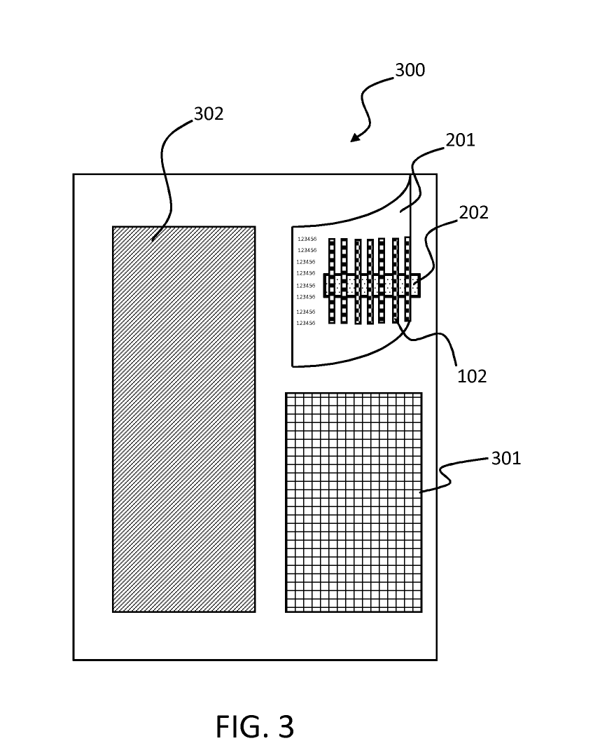

FIG. 3 is a top view of an embodiment of the sheet 200 illustrated in FIG. 2 with parts of the testing device 100 from FIG. 1. The sheet 300 illustrated in FIG. 3 is one particular embodiment of the sheet 200. Other embodiments are possible. As seen in FIG. 3, multiple strips 102 from a testing device, such as the testing device 100 of FIG. 1, have been attached to the adhesive strip 202 on sheet 300. These strips 102 can be read or interpreted to determine testing results. The shield 201 may be closed over the strips 102. Information may be marked in the table 301 beneath the shield 201. Additional information may optionally be included in the area 302 or other areas of the sheet 300. The scanner may read or digitize some or all of the sheet 300 or some or all of the data included therein.

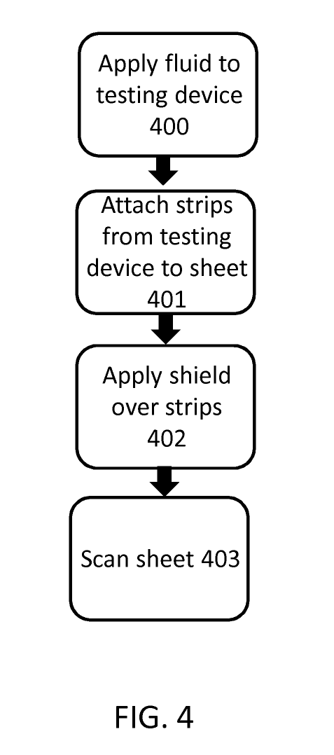

FIG. 4 is a flowchart of an embodiment of a method of using a testing device, such as the testing device 100 of FIG. 1. In step 400, a fluid is applied to at least part of a testing device, such as a strip 102 from FIG. 1. The testing device may have multiple antigens and controls on strips of the testing device. Antigens on the testing device may react to this fluid. In step 401, at least the strips of the testing device are applied or otherwise disposed or attached to a sheet. In step 402, a shield affixed to the sheet covers the strips such that the strips are disposed between the sheet and the shield. In step 403, the sheet is scanned to read or digitize the data on the sheet or strips or to read or digitize some or all of the sheet or strips. Scanning may include OCR of data on the sheet or strips.

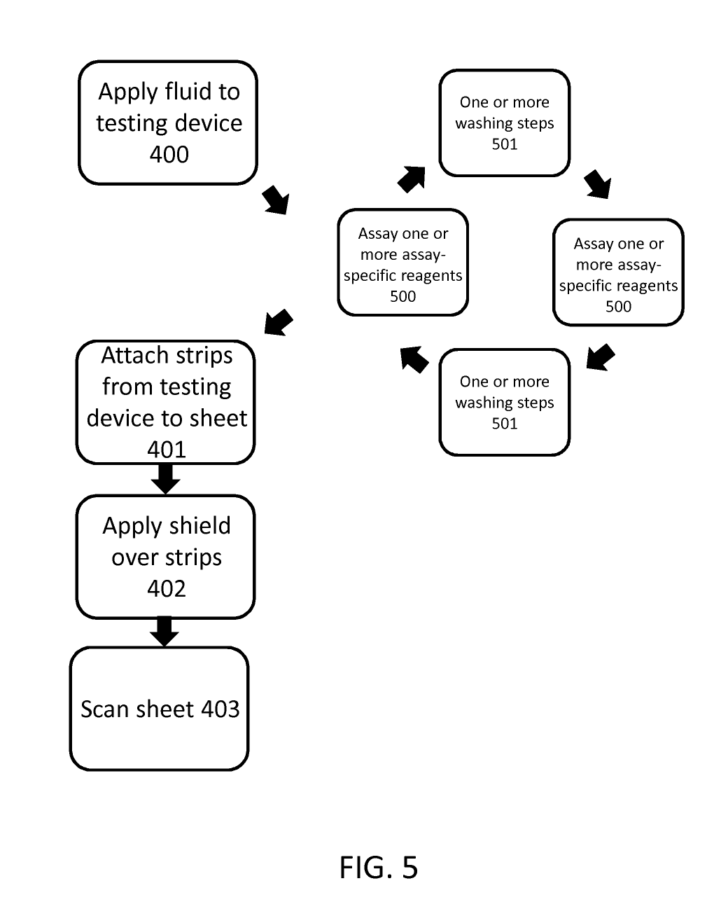

FIG. 5 is a flowchart of an exemplary method for using a testing device, such as the testing device 100 of FIG. 1. In this embodiment, a series of washing steps 501 and assays of assay-specific reagents 500 occurs between application of the fluid to the testing device 400 and attaching the strips to the sheet 401. Other variations are possible and this is merely an example. The exact number of assays 500 or washing steps 501 may vary.

It will be recognized from the foregoing that advantages over previously available technologies provided by the present disclosure include but are not necessarily limited to combining the capability for modular antigen application and assembly in individual strip format; a tracking line for use prior to a blocking step; complete traceability of strips with, for example, lot and individual strip indicia on each individual strip in an alpha-numeric or bar-code or pictographic format; a serum control (in addition to the sample) combined with an assay control on the same strip (specific to the function of other assay components); potential antibody isotype specificity; semi- or fully-quantitative capabilities; integration with supporting software and instrumentation, such as imaging software, densitometry and the like for normalizing assay data against control data; multi-session/multi-use report sheet; and highly customizable capability for comprehensive antigen panels tailored for any one or any constellation of auto-immune conditions/antigens, or any other antigens for which detection of an immune response (or lack thereof) is desirable.

Antigens that are provided with various embodiments of this disclosure, such as on the testing areas 105/antigen locations 106, can be any antigen(s). In general, the antigens are biological macromolecules such as peptides, polypeptides, nucleic acids, lipids, or carbohydrates. The antigens may also comprise natural or laboratory-derived complexes of these biological macromolecules. The antigens may also comprise carbohydrates and/or phospholipids. The antigens may also comprise polynucleotides, such as DNA or RNA. In embodiments, the antigens are autoimmune antigens. The antigens can be attached to the antigen locations 106 using any suitable technique, many of which are known to those skilled in the art and are used routinely for adhering antigens to substrates for immunological detection, such as for Western blotting. Such techniques can be modified depending on whether the antigen is a protein, or a lipid-based antigen, or for example a polynucleotide antigen, and can accordingly be formed on distinct substrates to take advantage of, for example, hydrophobic interactions between the antigen and the substrate used to form the antigen location 106. In embodiments, the antigens are contacted with the antigen locations for a period of time such that the antigen is reversibly or irreversibly attached to the antigen location. In non-limiting embodiments, the antigens are autoimmune antigens and are selected from antigens to which antibodies in a biological sample specifically bind are indicative of any of the following conditions: Addison's Disease, Antiphospholipid Syndrome, Autoimmune Carditis , Autoimmune Gastritis, Autoimmune Hepatitis, Autoimmune Neuropathies, Autoimmune Parathyroid Disease, Cardiovascular Disease, Celiac Disease, Churg-Strauss Syndrome, Crohn's Disease, Dermato/Polymyositis, Dermatopathology, Diabetes Type 1, Glomerulonephritis, Goodpasture syndrome, Graves Disease, Hashimoto's Disease, Infertility, Inflammatory Bowel Disease, Myasthenia Gravis, Neuropsychiatric Lupus, Ocular Pathology, Oral Pathology, Paraneoplastic Syndromes, Pernicious Anemia, Polyarteritis Nodosa, Primary Biliary Cirrhosis, Primary Sclerosing Cholangitis, Relapsing Polychodritis, Rheumatoid Arthritis, Scleroderma, Sensorineural Hearing Loss, Sjögren's Syndrome, Systemic Lupus Erythematosus (SLE), Ulcerative Colitis, Wegener's granulomatosis, and combinations thereof.

In particular embodiments, the antigens 106 provided with embodiments of the present disclosure may be antigens defined by being specifically recognized by any of the following antibodies: Anti-Adrenal Antibodies, Anti-Asialo GM1 Antibodies, Anti-Basement Membrane Zone Antibodies (BMZ), Anti-Centromere, Anti-Cyclic Citrullinated Peptides Antibodies (CCP), Anti-dsDNA Antibodies, Anti-Endomysial Antibodies (EMA), Anti-GAD, Anti-Galactocerebroside Antibodies, Anti-Ganglioside Antibodies, Anti-Gastric Parietal Cell Antibodies (AGPA), Anti-GD1a Antibodies, Anti-GD1b Antibodies, Anti-Gliadin Antibodies (AGA), Anti-Glomerular Basement Membrane (GBM) Antibodies, Anti-GM1 Antibodies, Anti-GQ1b Antibodies, Anti-Heart Antibodies, Anti-Histone Antibodies, Anti-Intercellular Antibodies (IC), Anti-Intrinsic Factor Antibodies, Anti-Islet Cell Antibodies, Anti-Jo-1 Antibodies, Anti-Keratin Antibodies, Anti-Liver/Kidney/Microsomal 1 (LKM-1) Antibodies, Anti-Microsomal Antibodies (TPO), Anti-Mitochondrial Antibodies, Anti-Mitochondrial M2 Antibodies, Anti-Myelin Associated Glycoprotein Antibody (MAG), Anti-Myeloperoxidase Antibodies (MPO), Anti-Myositis Antibodies, Anti-nDNA Antibodies (Crithidia Luciliae), Anti-Neuronal Antibodies (Hu, Yo, Ri & Tr), Anti-Neutrophil Cytoplasmic Antibodies (ANCA), Anti-Nuclear Antibodies (ANA), Anti-Oxydized LDL Antibodies (OxLDL), Anti-PO Antibodies (Protein Zero), Anti-Phospholipid/Cardiolipin Antibodies (APL), Anti-Pm/Sc1 Antibodies, Anti-Proteinase 3 Antibodies (PR-3), Anti-Reticulin Antibodies (ARA), Anti-Rheumatoid Factor (RF), Anti-Ribosomal P Antibodies, Anti-RNA Antibodies, Anti-RNA Polymerase, Anti-RNP Antibodies, Anti-Saccharomyces Cerevisiae Antibodies (ASCA), Anti-Sc1-70 Antibodies, Anti-Skin Antibodies, Anti-Sm Antibodies, Anti-Sm/RNP Antibodies, Anti-Smooth Muscle Antibodies (ASMA), Anti-β2-Glycoprotein Antibodies (β2-GP1), Anti-SS-A (Ro) Antibodies, Anti-SS-B (La) Antibodies, Anti-ssDNA Antibodies, Anti-Steroidal Antibodies, Anti-Striational Muscle Antibodies, Anti-Thyroglobulin Antibodies (Tg), Anti-Tissue Transglutaminase (tTG) Antibodies, Disease Association/Pharmacogenetics, Exocrine Pancreas Antibodies (ExPA), Extractable Nuclear Antigen (ENA) Antibodies. Embodiments of the invention include panels that comprise any combination of antigens that are defined at least in part by being specifically recognized by these antibodies. Those skilled in the art will recognize that the antigens are accordingly defined by antibodies which recognize them. As one non-limiting embodiment, the disclosure of Anti-Tissue Transglutaminase (tTG) Antibodies indicates that an antigen that can be provided with embodiments of the present disclosure is tTG. The antigens can be attached to the antigen locations 106 using any suitable reagents and techniques. In embodiments, the antigens are non-covalently or are covalently attached to the antigen location 106. In embodiments, the antigens are cross-linked to a substrate that is present in antigen location 106.

In general, the present disclosure includes the foregoing embodiments, but also methods of using such embodiments. Typically, each strip 102 and/or testing area 105 is contacted with a biological sample obtained from an individual. The biological sample can be used directly, or it can be subjected to a processing step, such as to isolate and/or purify a portion of the sample which is likely to comprise antibodies, or to concentrate antibodies in the biological sample. The biological sample can be a biological fluid. The biological sample can be mixed with any suitable buffer so that it can be exposed to the strip 102.

In one example, the strip 102 or testing device 100 is incubated in a blocking buffer to inhibit non-specific background binding of the antibodies. Any suitable blocking buffer can be used are many such solutions are commercially available or can be prepared by the testing laboratory using routine techniques. The sample is typically rinsed after blocking with a suitable buffer, and may or may not be dried to be prepared for sample addition. The sample is added, and the strip 102 or testing device 100 is incubated with the sample for conventional period of time and at a standard temperature and washed. Subsequent incubations with enzyme antibody conjugate or substrate interspersed by wash steps may be performed as needed and according to immunodetection methods that are known in the art. The dispensation of reagents (including the sample) can be manual, such as with a pipette, or automatic. Automatic dispensation can use, for example, instruments such as Tecan Profiblot, DAS Speedy line of instruments, Bee Blot Processor by Bee Robotics, or TrinBlot processor by Trinity Biotech. Following washing and drying, the testing device 100 or strip 102 is interpreted by a technician or automated method such as software coupled with a scanner or camera.

The invention includes detecting antigen/antibody complexes bound to the strips, as well as a lack of antigen/antibody complexes, and generating a report based on said detection. This may include determining at least one of the presence, absence, or amount of a complex or macromolecule. The report can be fixed in a tangible medium of expression, such as an electronic file, printed material, or any electronic storage medium. The report can be communicated to a health care worker, a laboratory, or an individual from whom the biological sample tested using embodiments of the present disclosure was obtained. In embodiments, the report is used for diagnosis of a disease. In embodiments, the report is a diagnosis, or aids in the diagnosis of a disease. In embodiments, the report aids in the diagnosis of any autoimmune disease described herein.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A testing device comprising:
a base;
a plurality of strips connected to said base, each of said plurality of strips having a long dimension and a short dimension that define a surface; and
a plurality of distinct antigens on said surface of each of said strips, wherein each of said antigens is disposed on each of said strips at a same position along said long dimension.

2. The testing device of claim 1, wherein each of said strips contains at least three different controls.

3. The testing device of claim 2, wherein each of said strips contains four of said different controls.

4. The testing device of claim 1, wherein each strip has an identification feature on said surface opposite said base.

5. The testing strip of claim 4, wherein said identification feature is a code unique to said strip that said identification feature is disposed on.

6. The testing device of claim 1, further comprising a plurality of testing areas on each of said strips, wherein each of said testing areas contains at least one of said antigens.

7. The testing device of claim 6, wherein said testing area contains at least two of said antigens.

8. The testing device of claim 1, wherein said base and said strips define perforations proximate an end of each of said strips closest to said base.

9. The testing device of claim 1, wherein said testing device is configured for line immunoassay testing.

10. A testing system comprising:
a testing device comprising:
a base;
a plurality of strips connected to said base, each of said plurality of strips having a long dimension and a short dimension that define a surface; and
a plurality of distinct antigens on said surface of each of said strips, wherein each of said antigens is disposed on each of said strips at a same position along said long dimension; and
a sheet comprising a shield, wherein at least said strips are configured to be affixed to said sheet under said shield.

11. The testing system of claim 10, wherein said sheet further comprises an adhesive strip configured to affix said strips to said sheet.

12. The testing system of claim 10, wherein said shield is permanently affixed to said sheet along at least one edge of said shield.

13. The testing system of claim 10, wherein said sheet further comprises a table on a surface of said sheet with said shield.

14. The testing system of claim 10, wherein said testing device is configured for line immunoassay testing.

15. A method comprising:
receiving a sheet of inert material that further comprises a plurality of antigen locations and control locations;
receiving a number of a plurality of strips to be formed in said sheet and dimensions for said strips;
cutting gaps in said sheet with a laser to form said number of said strips with said dimensions, each of said strips having a long dimension and a short dimension that define a surface, said antigen locations and said control locations being disposed on said surface of each of said strips, wherein a positional order of said antigen locations and said control locations is identical along said long dimension of each of said strips, said strips being connected to a base; and
forming an identification feature on said surface of each of said strips using said laser.

16. The method of claim 15, wherein said inert material is plastic.

17. The method of claim 15, further comprising forming perforations in said sheet between said strips and said base using said laser.

18. The method of claim 15, wherein said identification feature is a code unique to said strip.

19. The method of claim 15, wherein said method forms a testing device configured for line immunoassay testing.
